(19) United States
(12) Patent Application Publication
(10) Pub. No.: US 2026/0060732 A1
(43) Pub. Date: Mar. 5, 2026

(54) ORTHOPEDIC IMPACTOR INCLUDING A SOLENOID ARMATURE POSITION SENSING SYSTEM

(71) Applicants: Smith & Nephew, Inc., Memphis, TN (US); Smith & Nephew Orthopaedics AG, Zug (CH)

(72) Inventors: Giorgos Karatziolas, London (GB); Ruben Doyle, London (GB)

(73) Assignees: Smith & Nephew, Inc., Memphis, TN (US); Smith & Nephew Orthopaedics AG, Zug (CH)

(21) Appl. No.: 19/311,535

(22) Filed: Aug. 27, 2025

Related U.S. Application Data
(60) Provisional application No. 63/690,507, filed on Sep. 4, 2024.

(51) Int. Cl. A61B 17/92; A61B 17/00

(52) U.S. Cl. CPC A61B 17/92; A61B 2017/0003; A61B 2017/924

(57) ABSTRACT

An orthopedic surgical impactor and corresponding methods of operations. The impactor includes an electromagnetic component having a stationary electromagnetic housing and a moving armature component. The stationary electromagnetic housing includes a coil configured to receive an electric current resulting in generation of an electromagnetic field for triggering translation of the armature component within the stationary electromagnetic housing. The electromagnetic field is configured force the armature component to translate in at least one direction. The impactor includes a sensing component having at least one processor. The processor is configured to determine an inductance, associated with the electromagnetic field, based on at least one of: at least one current output signal and at least one voltage output signal generated by the coil, and determine, based on the inductance, a position of the movable armature component within the stationary electromagnetic housing.

### CROSS-REFERENCE TO RELATED APPLICATIONS

This is a non-provisional of, and claims the benefit of the filing date of, pending U.S. Provisional Patent Application No. 63/690,507, filed Sep. 4, 2024, entitled “Orthopedic Impactor Including a Solenoid Armature Position Sensing System,” the entirety of which application is incorporated by reference herein.

### TECHNICAL FIELD

The present disclosure is generally directed to a surgical impactor, and, in particular, to an orthopedic surgical impactor having an armature position sensing system.

### BACKGROUND

Orthopedic surgical procedures such as, for example, hip procedures, knee procedures, shoulder procedures, etc., have become common place in today's society. For example, total hip arthroplasty or hip replacement is a well-known procedure for repairing damaged bone (e.g., a damaged hip). During a total hip arthroplasty, an acetabular system may be implanted into a patient's acetabulum. In addition, and/or alternatively, a femoral implant may be implanted into a patient's femur. During the surgical procedure, the patient's bone typically needs to be prepared to receive the orthopedic implant. For example, a surgical tool such as, for example, an orthopedic broach, rasp, cutting tool, etc. (terms used interchangeably herein without the intent to limit or distinguish) may be used to prepare an inner surface of a patient's intramedullary canal to receive an orthopedic implant such as, for example, a femoral hip prosthesis, an intramedullary nail, etc. The preparation of the intramedullary canal by the surgeon is designed to ensure a proper fit between the patient's femur and the implant. In addition, the orthopedic implant such as, for example, the acetabular cup, may need to be impacted into proper position. Moreover, during removal of the broach from the patient's intramedullary canal, the broach may become stuck within the patient's intramedullary canal.

A surgical impactor may be used for insertion and/or removal of implants. Some existing impactors rely on motors to deliver energy for such insertion/removal. Other impactors us solenoid-based systems that produce electromagnetic energy that is applied to deliver the necessary force to objects coupled to the impactor (e.g., tools, implants, etc.), and hence, perform requisite surgical procedures. The solenoid-based impactor systems may require multiple firings (e.g., applications of current to the solenoid) to fire the solenoid in both a forward and reverse direction of operation to ensure that force is applied in the direction desired by the surgeon. This reduces effectiveness of the impactor operations during surgical procedures by consuming excess power and causing unnecessary impacts to be delivered. As such, it would be beneficial to provide a surgical impactor tool that eliminates such multiple firings and determines intended direction of operation prior to delivery of an impact by the impactor.

### SUMMARY

This summary is provided to introduce a selection of concepts in a simplified form that are further described below in the detailed description. This summary is not intended to identify key features or essential features of the claimed subject matter, nor is it intended as an aid in determining the scope of the claimed subject matter.

In some examples, the present disclosure relates to an orthopedic surgical impactor and methods of operation thereof. The impactor may include an electromagnetic component including a stationary electromagnetic housing and a moving armature component. The stationary electromagnetic housing may include a coil configured to receive an electric current resulting in generation of an electromagnetic field for triggering translation of the armature component within the stationary electromagnetic housing. The impactor may also include a sensing system and/or component(s) (terms used interchangeably herewith). The sensing system/component may include at least one processor that may be configured to determine an inductance, associated with the electromagnetic field. The inductance may be determined based on at least one of: at least one current output signal and at least one voltage output signal generated by the coil. Using the inductance, the processor may further determine a position of the movable armature component within the stationary electromagnetic housing.

Examples of the present disclosure provide numerous advantages. For example, the current subject matter's determination of inductance associated with the coil may accurately determine intended direction of operation of the impactor (e.g., reverse (e.g., for removal of an implant) and/or forward (e.g., for setting of an implant)). Such determination may be based on output signals generated by the coil, thereby avoiding incorporation of additional electronic components, wiring, and/or circuitry. Moreover, accurate determination of intended direction of operation of the impactor may reduce undesired movement of the distal connector of the impactor, which may affect the intent and result of the surgical procedure. Further, the current subject matter may prevent occurrence of double firing of an impactor during operation, where the impactor fires in one direction to prime the armature component before it can be operated in an alternate direction. Such double firing substantially increases power consumption by the impactor, thereby reducing effectiveness of its operation.

Further features and advantages of at least some of the examples of the current subject matter, as well as the structure and operation of various examples of the current subject matter, are described in detail below with reference to the accompanying drawings.

### BRIEF DESCRIPTION OF THE DRAWINGS

The accompanying drawings, which are incorporated in and constitute a part of this specification, show certain features of the subject matter disclosed herein and, together with the description, help explain some of the principles associated with the disclosed implementations. In the drawings,

FIG. 1 illustrates an exemplary orthopedic surgical impactor that may be configured to incorporate an armature position sensing system, in accordance with one or more features of the present disclosure;

FIG. 2A illustrates the armature component of the impactor shown in FIG. 1 prior to operation in one direction (e.g., a “pushing” or “forward” direction);

FIG. 2B illustrates the armature component of the impactor shown in FIG. 1 prior to operation an alternate direction (e.g., “pulling” or a “reverse” direction);

FIG. 3 illustrates an example controller that may be incorporated into the electronics of the impactor shown in FIG. 1 in accordance with one or more features of the present disclosure;

FIG. 4 is a plot illustrating an example of a linear relationship between the reciprocal inductance and the position of the armature component of the impactor shown in FIG. 1 in accordance with one or more features of the present disclosure;

FIG. 5 is a block diagram of an exemplary orthopedic surgical instrument and/or impactor in accordance with one or more features of the present disclosure;

FIG. 6 illustrates an exemplary computing apparatus in accordance with one or more features of the present disclosure;

FIG. 7 illustrates an example of a storage medium to store impactor logic in accordance with one or more features of the present disclosure; and

FIG. 8 illustrates an example computing platform in accordance with one or more features of the present disclosure.

It should be understood that the drawings are not necessarily to scale and that the disclosed examples are sometimes illustrated diagrammatically and/or in partial views. In certain instances, details that are not necessary for an understanding of the disclosed methods and devices or which render other details difficult to perceive may have been omitted. It should be further understood that this disclosure is not limited to the particular examples illustrated herein. In the drawings, like numbers refer to like elements throughout unless otherwise noted.

### DETAILED DESCRIPTION

To address these and potentially other deficiencies of currently available solutions, one or more implementations of the current subject matter relate to methods, systems, articles of manufacture, and the like that can, among other possible advantages, provide an orthopedic surgical instrument or impactor, and in particular, a surgical impactor tool that may include an armature position sensing system for detecting position of an armature.

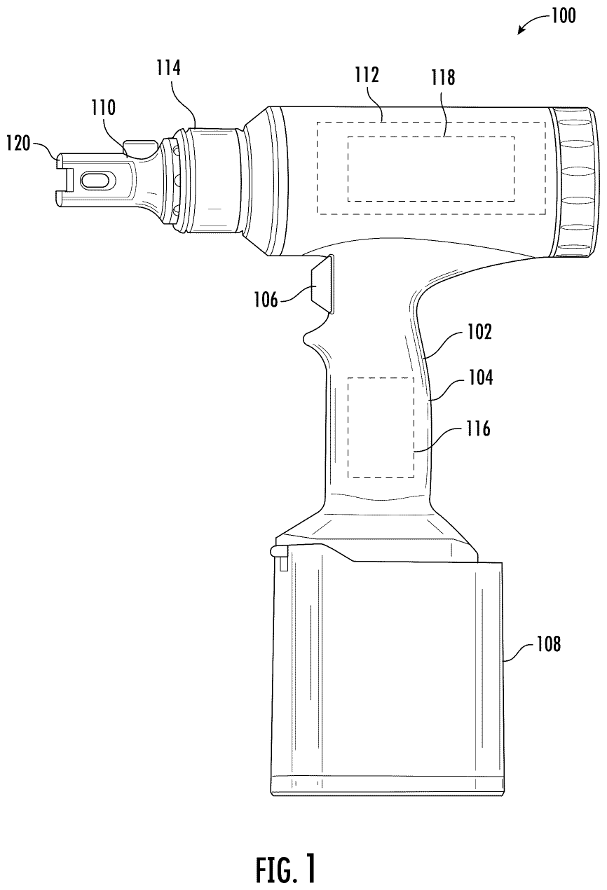

It should be appreciated that while, for example, detection of whether a forward or a reverse impact is desired in accordance with the present disclosure may be described herein in connection with an orthopedic surgical impactor (e.g., as shown in FIG. 1) that may be used to, for instance, drive a broach into a patient's bone to, for example, prepare an intramedullary canal of the patient's bone, the present disclosure is not so limited and such detection and subsequent operation (as shown and described hereinbelow) may be used in connection with any tool, device, instrument, etc. now known or hereafter developed. As such, the present disclosure should not be limited to any particular tool unless explicitly claimed.

The following description of an orthopedic impactor (e.g., as shown in and discussed in connection with FIG. 1) is provided here for exemplary, illustrative purposes only and is not intended to limit the current subject matter and/or any of its elements, applications and/or advantages.

With reference to FIG. 1, an example of an orthopedic surgical instrument, impactor, or impactor mechanism (terms used interchangeably herein without the intent to limit or distinguish) is disclosed. The orthopedic impactor is arranged and configured to transmit a forward energy or motion to, for example, drive a surgical tool (e.g., a broach) or implant into a patient's bone, and a reverse energy or motion to, for example, remove a stuck or lodged surgical tool (e.g., a broach) or implant from a patient's bone.

The orthopedic impactor may be arranged and/or configured to position, insert and/or implant an orthopedic implant such as, for example, but not limited to, an acetabular cup, an intramedullary nail, a femoral hip implant, etc. into a bone matter (e.g., a bone of a patient). Alternatively, or in addition to, the orthopedic impactor may be coupled to a surgical tool such as, for example, but not limited to, a broach, to prepare a bone to receive an orthopedic implant.

The orthopedic impactor may cause application of a forward energy and/or motion to drive an orthopedic implant and/or surgical tool into a patient's bone. In addition, the orthopedic impactor may be arranged and/or configured to cause application of a reverse energy and/or motion to, for example, but not limited to, remove a stuck and/or lodged surgical tool and/or implant from a patient's bone. The orthopedic impactor may be configured to allow selection between the forward and/or reverse application of energy and/or motion by pushing forward and/or pulling back on the orthopedic impactor. A user (e.g., a medical professional, a doctor, a surgeon, etc.) may push forward on an orthopedic impactor thereby causing a hammer of the impactor to strike a first and/or a forward impaction surface causing the orthopedic impactor to drive an orthopedic implant and/or surgical tool. Alternatively, or in addition, the user may pull back on the orthopedic impactor thereby causing the impactor's hammer to strike a second and/or a reverse impaction surface causing the orthopedic impactor to produce a reverse impaction to, for example, remove an orthopedic implant or surgical tool.

The orthopedic impactor may be arranged and/or configured to apply a force, while minimizing the risk of injury to the patient and/or to the user's hands during use. Moreover, the orthopedic impactor may be configured to assist its user to deliver a force towards and/or away from a surgical area in, for example, but not limited to, a joint replacement procedure.

In some implementations, the current subject matter relates to an impactor that may incorporate electromagnetic devices and/or systems. The impactor may include an electromagnetic component having a stationary electromagnetic housing and a moving armature component. The stationary electromagnetic housing may include one or more coils. The coil(s) may be configured to receive an electric current. The armature component may be manufactured from one or more magnetic materials and may be configured to generate a magnetic field that may interact with the electric current applied to the coil(s) and trigger translation movement of the armature component.

The impactor may also include a sensing system and/or component(s) (terms used interchangeably herewith). The sensing system/component may include at least one processor that may be configured to determine an inductance, associated with the electromagnetic field. The inductance may be determined based on at least one of: at least one current output signal and at least one voltage output signal generated as a response to a signal (e.g., generated by a processor) from the coil. Using the inductance, the processor may further determine a position of the movable armature component within the stationary electromagnetic housing. In some implementations, the processor may determine the coil's inductance based on a plurality of current output signals and/or a plurality of voltage output signals generated as a response to a signal by the coil. The inductance may be a reciprocal inductance (i.e., an inverse of the inductance) and may be used to define a linear relationship with the position of the armature component. The position of the movable armature component may be determined based on a distance that the movable armature component translated within the stationary electromagnetic housing. Using the position, the processor may determine an intended direction of operation of the impactor. The intended direction may include, for example, at least one of: a first forward direction (e.g., a “pushing” or “forward” direction) of operation of the impactor and a second operation (e.g., a “pulling” or “reverse” direction) of the impactor. As can be understood, any directions of operation are possible. The terms “pushing,” “forward,” “pulling,” and “reverse” are arbitrary and are used herein for illustrative, non-limiting and ease of description purposes only. In some implementations, upon the distance from a zero point being greater than or equal to a predetermined threshold distance (e.g., 3 mm), the processor may determine that the intended direction is the forward direction. Alternatively, or in addition, upon the distance being less than a predetermined threshold distance, the processor may determine that the intended direction is the reverse direction.

The impactor may further include a striker body coupled to the armature component and an object (e.g., an implant, a tool, a connector, etc.), where the striker body may include a distal end. The impactor may also include a strike chamber having an interior and a strike wall disposed at at least one end of the strike chamber.

In some implementations, an electromagnetic field may be generated as a result of application of the electric current to the coil. The generated electromagnetic field may force the armature component and the striker body to translate. Translation of the armature component causes the distal end of the striker body to strike a strike wall.

In some implementations, the directions of translation movement of the armature component and the striker body may be interdependent, which in turn, may be dependent on a direction of the current applied to the coil(s) within the stationary electromagnetic housing (once the intended direction of operation is ascertained). Further, the direction of translation movement of the armature component and the striker body may be directly and/or indirectly dependent on the direction of the current. Further, a change in the direction of the current applied to the armature component may change the direction of translation movement of the armature component and the striker component. In some implementations, a single coil and/or a set of coils may be configured to be used for translating the armature component in both directions. Alternatively, or in addition, one coil and/or one set of coils may be configured to be used for translating the armature component in one direction and another coil and/or another set of coils may be configured to be used for translating the armature component in another (and/or opposite) direction. As can be understood, any combination of coils may be used for translating the armature component and/or changing direction of translation.

FIG. 1 illustrates an exemplary impactor 100 that may be configured to incorporate a system for detecting whether a forward or a reverse impact is desired and, as a result, operating the impactor (as shown and described below), in accordance with one or more features of the present disclosure. The impactor 100 may be configured to include a housing 102, a handle 104, an actuator or a trigger assembly 106, a power source 108, a distal connector assembly 110, a strike mechanism housing 112, a distal connector housing 114, electronics 116, and a strike mechanism 118 (positioned within the strike mechanism strike mechanism housing 112). In some example, non-limiting implementations, the impactor 100 may also include a user interface, an electronic display and/or an input keypad and/or key panel (e.g., touch-based keypad/key panel) that may be used to display and/or allow the user to enter various operational parameters of the impactor 100.

The housing 102 may be configured to house and/or enclose one or more components of the impactor 100. The housing 102 may be manufactured from any suitable material now known or hereafter developed such as, for example, but not limited to, plastic, metal, composite material, fiberglass, and/or any combination thereof.

The housing 102 may include the handle 104 with an optional handgrip for comfortable and secure holding of the impactor 100 during a procedure (e.g., positioning of an implant into a bone). Alternatively, or in addition, the housing 102 may incorporate a suitable mounting interface for integrating the impactor 100 into a robotic assembly during use. In some example implementations, the housing 102 may be a unitary structure and/or may include multiple components that may be assembled together.

The housing 102 may also include a reception port for receiving the power source or the battery (hereinafter, “battery”) power source 108. The power source 108 may be a rechargeable battery and may be removed from the housing 102 after use, such as, for example, for recharging. As can be understood, the power source 108 may recharged while coupled to the housing 102. The power source 108 may provide for portability and versatility of the impactor 100 during use, i.e., the user of the impactor 100 does not have to be concerned with power wires (and/or pneumatic tubes) extending from the impactor 100. Alternatively, or in addition, the housing 102 may include one or more power ports (not shown in FIG. 1) that may be used to couple one or more power wires (e.g., to provide power and/or power in addition to the power source 108) and/or one or more pneumatic tubes (e.g., to provide additional air pressure to the impactor 100 during use). As can be understood, more than one power source 108 may be included in the housing 102 and/or used during procedures. Any type of battery may be used, such as, for example, but not limited to, alkaline, nickel metal hydride (NiMH), lithium ion, and/or any combination thereof. The power source 108 may be incorporated and/or inserted into the handle 104 and/or coupled to the handle 104 (FIG. 1 illustrates the power source 108 being incorporated and/or inserted into the handle 104). As can be understood, other ways of coupling and/or connecting the power source 108 to electrical circuits of the impactor 100 are possible.

In some example, non-limiting implementations, the power source 108 may be at least one of the following: a capacitor, a supercapacitor, an interface for receiving mains power, an external power source, a chemical power source, and/or any combinations thereof. Further, combining a battery and/or a capacitor/supercapacitor may enable provision of a large power source that may, via charging of a capacitor, provide a large instantaneous and/or short-term current.

The power source 108 may be configured to provide current to power the electronics 116, which may be used to control operation of the impactor 100. The electronics 116 may be disposed in the handle 104, the strike mechanism housing 112, and/or both of the impactor 100. The power source 108 may be coupled to the electronics 116 using one or more wires. Alternatively, or in addition, the power source 108 may wirelessly (e.g., using near field) supply power to the electronics 116. The electronics 116 may include a central processing unit (CPU) that may be arranged and configured to generate and/or execute one or more instructions associated with operation of the impactor 100. The electronics 116 may also include a memory for storing various data, information, etc., such as, for example, instructions for the CPU, a permanent storage for storing larger amounts of information, a communications interface for communicating with one or more components of the impactor 100 or with one or more external devices (e.g., a USB type port, a network connector, etc.), and/or any other components. In some example, alternate implementations, the impactor 100 and/or the electronics 116 may include one or more microcontrollers that may be arranged and configured to control and/or coordinate one or more components of the impactor 100.

In some implementations, as stated above, the impactor 100 may include one or more user interfaces (not shown in FIG. 1) that may be used for inputting various operational parameters and/or changes thereof. For example, the parameters may include, but are not limited to, a speed of an impact, a force of the impact, a frequency of the impact, a direction of the impact (e.g., whether to drive in an implant or remove an implant), a depth of the implant (e.g., how far into a bone the implant is to be driven), a stability of the implant, and/or any other parameters. These parameters may relate to a single impact and/or to multiple impacts. In some implementations, the user may input a single parameter, using which, the electronics 116 may be configured to determine any other operations parameters.

In some implementations, the user interface may be used for not only inputting operational parameters of the impactor 100, but also for observing its operation and viewing how changes in one or more operational parameters may affect operation of the impactor 100. For example, a change in frequency of the impact may trigger changes (e.g., automatic changes and/or a request for manual changes) in force of the impact and a speed of the impact. The electronics 116 may determine such changes and display changed parameters, along with an any effects on the operation of the impactor 100 and/or implantation/removal process, on the user interface. For example, the effects may include an indication of a change in the remaining battery level, and/or a determined impact stability, a total impact energy that has been transferred to the implant, etc.

In some example, non-limiting implementations, the strike mechanism 118 may be used to actuate positioning and/or removal of an implant by the impactor 100 after receiving one or more instructions from the electronics 116. For example, upon actuating the trigger assembly 106 (e.g., after entry of appropriate operational parameters), the electronics 116 may be configured to generate one or more operational instructions in accordance with one or more operational parameters and provide such instructions to the strike mechanism 118. Upon receiving instructions from the electronics 116, the strike mechanism 118 may be configured to impart force to the distal connector assembly 110 (positioned within the distal connector housing 114), which, in turn, may transfer that force to an object (e.g., an implant, a tool, etc.) that may be coupled to the distal connector assembly 110 and/or directly to the implant (either for positioning or removal).

In some implementations, the distal connector assembly 110 may include one or more attachment structures and/or mechanism 120 that may be arranged and configured to couple to an object (e.g., drills, cutting tools, effectors, broaches, implants, etc.). For coupling to the distal connector assembly 110, the object (not shown in FIG. 1) may include a click-fit, snap-fit, etc. attachment structure into which the distal connector assembly 110 may mate with. Alternatively, or in addition, the distal connector may include one or more of the following attachment structures and/or mechanisms: a gripping structure (e.g., a clamp), a snap-fit structure, an interference-fit structure, a magnetic attachment mechanism, a releasable attachment mechanism, an interchangeable attachment structure/mechanism, and/or any other type of structures/mechanisms. In some implementations, the distal connector assembly 110 may include a single attachment structure/mechanism and/or multiple attachment structures/mechanism, such as, for example, a snap-fit structure that may be used when implanting an object and a gripping structure that may be used for removing an object. The distal connector assembly 110 may be arranged and configured to be used with a plurality of attachment structures/mechanism may also be beneficial since manufacturers of different implants may require different attachment structure.

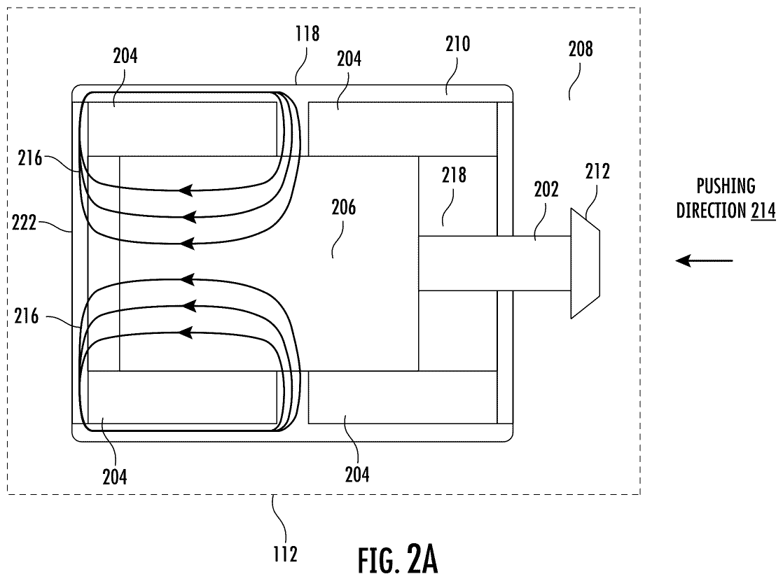

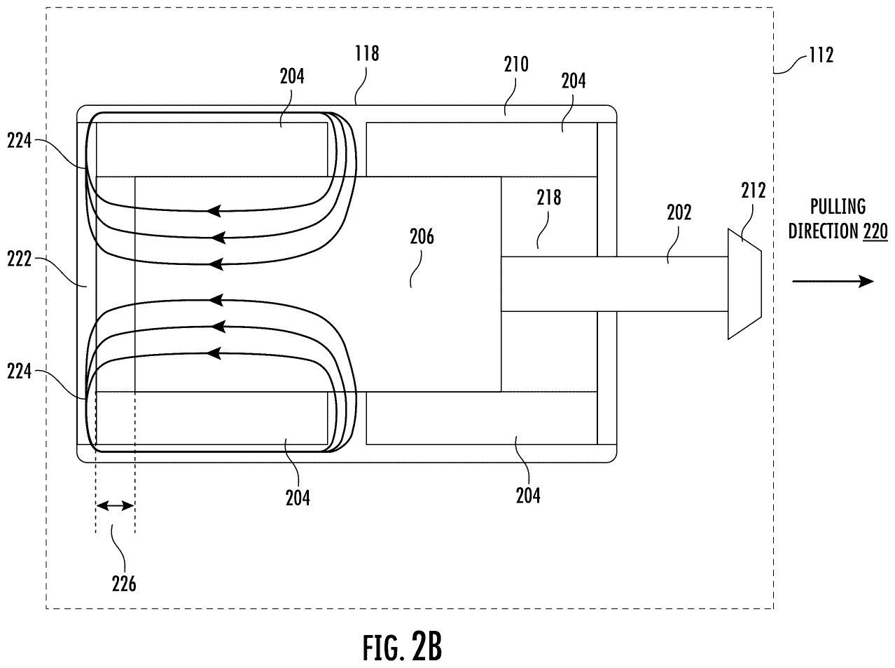

FIGS. 2A-B illustrate further details of the impactor 100 shown in FIG. 1, and in particular, operation of impactor's armature component 206, in accordance with one or more features of the present disclosure. Specifically, FIG. 2A illustrates pushing or forward operation of the armature component 206, which may, for instance, be used during positioning of an implant into a bone of a patient. FIG. 2B illustrates pulling or reverse operation of the armature component 206, which may, for example, be used during removal of an implant from a bone of a patient.

As stated above, the strike mechanism 118 may be used to interact with the object coupled to the distal connector assembly 110 (e.g., using structure(s)/mechanism(s) 120) for the purposes of imparting force to the object. The force may, for example, be used to insert an implant into a bone of a patient, and/or to remove an implant from the bone (e.g., to replace it, to correct improper installation, etc.). As can be understood, the force imparted by the strike mechanism 118 may be used for any other purposes. The strike mechanism 118 may be configured to impart force as a result of receiving current signals from electronics 116. The current signals may be used to generate an electromagnetic field triggering movement of at least one component of the strike mechanism 118, thereby causing the distal connector assembly 110 to translate.

As shown in FIG. 2A, the strike mechanism 118 may be disposed within an interior portion 208 of the strike mechanism housing 112. The interior portion 208 may be at least partially hollow so that it may accommodate positioning of the strike mechanism 118. The strike mechanism 118 may include a striker body 202 that may be positioned and configured to translate within a strike chamber (not shown in FIG. 2A), a stationary electromagnetic housing 210, an armature component 206 secured to the striker body 202, one or more coil(s) 204 that may be positioned around the armature component 206 and may be configured to interact with the armature component 206, which may be manufactured from one or more materials (e.g., steel, etc.) that may respond to application of a magnetic field. Alternatively, or in addition, the armature component 206 may include one or more magnets (e.g., permanent magnets).

The stationary electromagnetic housing 210 may be configured to remain stationary during operation of the impactor 100, while the armature component 206, along with the striker body 202, moves within an interior of the stationary electromagnetic housing 210 as a result of application of current. During operation of the impactor 100, the coil(s) 204 may receive electrical current from a battery (e.g., the power source 108, as shown in FIG. 1). In some example implementations, the stationary electromagnetic housing 210 may circumferentially surround the armature component 206 as well as the coil(s) 204 and the striker body 202. The coil(s) 204 may be configured to form a solenoid. One or more coil(s) 204 may be independent of one another (e.g., separately connected to and configured to receive current from the power source 108).

The stationary electromagnetic housing 210 may also include a hollow channel 218 that may accommodate positioning and translational movement of the armature component 206 while being secured to the striker body 202. The armature component 206 may translate within the hollow channel 218 between strikes performed by the striker body 202 after application of electrical current to the coil(s) 204. As stated above, the armature component 206 may be manufactured from material(s) that may respond to application of a magnetic field. Alternatively, or in addition, the armature component 206 may include one or more magnets, such as, for example, permanent magnets. As can be understood, the magnets may be any other types of magnets.

If magnets are used, each magnet may, by way of a non-limiting example, be configured to have a circular shape with an open interior portion (e.g., which may be similar to magnets having a donut shape). Outer diameters of the magnets may be the same and/or different. Similarly, the inner diameters of open interior portions of the magnets may be the same and/or different. In some example implementations, by having the magnets have same outer diameters and same inner diameters of their respective interior portions may allow for smoother translation movements of the armature component 206 within hollow channel 218. The magnet polarities' arrangement may be configured to generate a high magnetic potential. As can be understood, any desired arrangement of magnets may be used.

In some implementations, no permanent magnets are used in the impactor 100. Instead, the armature component 206 may be ferromagnetic (e.g., 430FR stainless steel, and/or any other material) and/or may include one or more portions that are manufactured from ferromagnetic materials. Ferromagnetic materials may refer to materials that exhibit strong magnetic properties and may be characterized by an alignment of magnetic moments in a common direction. When the materials are exposed to an external magnetic field, they become magnetized and maintain such magnetization after removal of the field. By way of a non-limiting examples, ferromagnetic materials that may be used for the armature component 206 and/or any of its portions may include iron, cobalt, nickel, their alloys, and/or any other materials, and/or any combinations thereof. Ferromagnetic materials for and/or in the armature component 206 may allow the armature component 206 to interact with the current supplied to the coil(s) 204 without the use of permanent magnets. This, in some example, non-limiting implementations, may reduce an overall weight of the armature component 206 and thus, the impactor 100.

Once current is supplied to the coil(s) 204, the coil(s) 204 may become energized. The energized coil(s) 204 may interact with the magnetic potential forcing translation of the armature component 206 to translate within the hollow channel 218. The direction of translation of the armature component 206 may depend on the direction of the current supplied to the coil(s) 204. By way of a non-limiting example, supplying the current in a counterclockwise direction in the coil(s) 204 may cause a forward translational movement of the armature component 206 causing the striker body 202 and distal connector assembly 110 to translate forward (e.g., in a pushing direction 214), and supply of the current in a clockwise direction in the coil(s) 204 may cause a reverse translational movement of the armature component 206 (i.e., in a pulling direction 220). As can be understood, the current subject matter is not limited to the above examples, and the current may be supplied to the coil(s) 204 in any desired direction so that the armature component 206 may be arranged and configured to move in a particular desired direction. Further, the “forward” and “reverse” designations are non-limiting and are provided herein for illustrative purposes only.

The coil(s) 204 may be arranged and configured to be positioned proximate to the armature component 206 and may further be configured to surround the armature component 206. Having the coil(s) 204 positioned proximate to the armature component 206 may allow for generation of an increased electromagnetic field, which, in turn, may allow for a more forceful translational movement of the armature component 206 (either forward or in reverse) as well as the striker body 202 and the distal connector assembly 110 (not shown in FIG. 2A).

The striker body 202 and, in particular, its distal end 212 may be configured to translate within the strike chamber upon application of electromagnetic force. The strike chamber may be a hollow space that allows the striker body 202 to translate within it. Once the electromagnetic force is generated as a result of an application current to the coil(s) 204, the armature component 206 and the striker body 202 may be configured to translate causing the distal end 212 of the striker body 202 to trigger translation of the distal connector assembly 110. This, in turn, transfers impact to any objects, tools, implants, etc. that may be coupled to the distal connector assembly 110. Once impact motion is completed, the striker body 202 and the armature component 206 may be returned to a pre-impact position (e.g., a spring may be positioned around striker body 202 triggering a recoil movement of the striker body 202 and armature component 206.

In some implementations, the impactor 100 may be configured to determine position of the armature component 206 prior to movement for delivery of an impact, during movement for delivery of the impact, during movement after delivery of the impact, and/or any other movement. To do so, the impactor 100 may be configured to include an impact direction sensing system that may be configured to detect a position of the armature component, which, in turn, may be used to determine a desired direction (e.g., forward/pushing, or reverse/pulling) in which the user of the impactor 100 is trying to deliver an impact. The sensing system may be configured to also allow the impactor 100 to tune energization of one or more coil(s) 204 to match the desired direction of application, thereby reducing the impactor 100 power consumption.

In some example, non-limiting implementations, the sensing system does not require any physical sensors and/or wiring. For instance, the sensing system may be configured to determine that a displacement is present between the distal end of the armature component 206 and a proximal wall 222 of the stationary electromagnetic housing 210. This determination may be made at any time (e.g., prior to delivery of impact, during impact delivery, after impact delivery, etc.). For example, during a reverse movement of the impactor 100 (as shown in FIG. 2B), the armature component 206 may be displaced forward by a predetermined distance from a predetermined reference point (e.g., an initial position of the armature). Such displacement of the armature component 206 may cause an alteration to the magnetic flux path within the solenoid, thereby leading to a change in an inductance of one or more coil(s) 204. The change in the inductance may be ascertained by the electronics 116, which may be indicative that the user of the impactor 100 wishes the impactor 100 to perform a reverse movement operation (e.g., to unseat/remove an implant from a bone). During a forward movement of the impactor 100 (as shown in FIG. 2A), the user of the impactor 100 may push on the impactor 100, thereby causing the armature component 206 to be is pressed firmly against the proximal wall 222 of the stationary electromagnetic housing 210.

As shown in FIG. 2A, during a forward movement, the user of the impactor 100 may have positioned the distal connector assembly 110 of the impactor 100 against an implant (not shown in FIG. 2A) and may have applied a pushing pressure on the implant. This may cause application of pressure on the distal end 212 of the striker body 202 in a pushing direction 214. This, in turn, forces the armature component 206 to be pressed against the proximal wall 222 of the stationary electromagnetic housing 210. Alternatively, or in addition, a resting position of the armature component 206 may include the armature component 206 being pressed against the proximal wall 222. This may be accomplished using, for example, a spring (not shown in FIG. 2A). While in the armature component 206 is in the resting position and/or being pressed against the proximal wall 222, the rearward coil(s) 204 may be configured to generate electromagnetic field 216, which, as shown in FIG. 2A, may surround the rearward coil(s) 204 and at least a portion of the armature component 206. In particular, the rearward coil(s) 204 may be surrounded by a continuous path of ferromagnetic material (e.g., material having a low magnetic reluctance). The electronics 116 (as shown in FIG. 1) may be configured to determine magnetic properties of the rearward coil(s) 204, i.e., inductance of the coil(s) 204, based on current(s) that may be applied to the coil(s) 204, which may be referred to as stimulating the coil(s) 204, while the armature component 206 is in the position shown in FIG. 2A. The inductance may be determined using the inductor equation below

$$L \frac{di}{dt} = υ \qquad ( 1 )$$

where i is current applied to and/or measured at the coil(s) 204, v is voltage applied to and/or measured at the coil(s) 204, and L is inductance.

The current and voltage values may be provided to the electronics 116, whose processing component may be configured to determine inductance. Using the determined current, voltage, and inductance, the processing component of the electronics 116 may then be configured to determine position of the armature component 206. Using the determined position of the armature component 206, the processing component of the electronics 116 may then determine that a forward operation of the impactor 100 (e.g., seating of the implant) is intended. In some example, non-limiting implementations of the impactor 100 that may include two coils (e.g., one for forward operation and one for reverse operation), the electronics 116 may be configured to execute computing logic using the following equation:

$$\frac{1}{{L}_{FWD}} - \frac{1}{{L}_{RWD}} \approx kx + c \qquad ( 2 )$$

where L_{FWD} and L_{RWD} are inductances of the forward coil and rearward coil calculated using Equation 1; x is the armature displacement (e.g., in millimeters); k and c are constants that are determined using one or more mechanical parameters of the forward coil and rearward coil (e.g., material selection, geometry, number of wire turns in each coil, etc.). In some example, non-limiting implementations, constants k and c may be experimentally determined (e.g., using one or more calibration measurements) and thereby determine a relationship linking displacement and measured inductance values. As can be understood, the current subject matter is not limited to the above equation of determining direction of operation of the impactor 100. For instance, a position of the armature component (from which direction of operation may be determined) may be determined using a weighted sum of all individual coil reciprocal inductances.

As shown in FIG. 2B, during a reverse movement, such as, for example, when removing or unseating an implant, the user of the impactor 100 may position the distal connector assembly 110 of the impactor 100 against the implant (not shown in FIG. 2B) and may apply a pulling force on the implant (e.g., via distal connector assembly 110). This may cause application of pressure on the distal end 212 of the striker body 202 in a pulling direction 220. Such pressure, may, in turn, force the armature component 206 to translate away from the proximal wall 222 of the stationary electromagnetic housing 210. Translation of the armature component 206 may create an air gap or a displacement 226 between the proximal wall 222 and the armature component 206.

While the armature component 206 is being translated away from the proximal wall 222 of the stationary electromagnetic housing 210, the rearward coil(s) 204 may generate electromagnetic field 224, which, as shown in FIG. 2B, may surround the rearward coil(s) 204, at least a portion of the armature component 206, and the displacement 226. Movement of the armature component 206 in the pulling direction 220 introduces the displacement 226 into the flux path of the electromagnetic field 224, which affects inductance. Specifically, an overall magnetic reluctance of the coil(s) 204 flux path(s) may be increased. High reluctance flux path(s) may result in a lower measured inductance (i.e., a ratio of magnetic flux to coil current). Using the inductance, the electronics 116 may be configured to determine a position of the armature component 206, which may be indicative of an intended impactor operation. For instance, when the armature component 206 is determined to be away from the proximal wall 222 of the stationary electromagnetic housing 210 at a predetermined threshold distance (e.g., 3 mm, and/or any other desired distance), the electronics 116 may determine that a reverse operation is intended by the user. Otherwise, when the armature component 206 is pressed against the proximal wall 222 of the stationary electromagnetic housing 210, the electronics 116 may determine that a forward operation is intended by the user.

In some implementations, to ascertain specific position of the armature component 206, the electronics 116 may be configured to stimulate coil(s) 204 by causing the power source 108 to apply voltage to the coil(s) 204 and measuring resulting currents. Using the voltage and the currents, the processing component of the electronics 116 may determine specific position of the armature component 206, and hence, determine intended operation of the impactor 100.

In some example, non-limiting implementations, the electronics 116 of the impactor 100 may be configured to determine position of the impactor using voltage and current data that it receives from the coil(s) 204. Alternatively, or in addition, one or more sensors may be incorporated into/onto and/or positioned in/on, for example, strike mechanism housing 112, hollow channel 218, armature component 206, striker body 202, proximal wall 222, and/or any other portion and/or component of the impactor 100. The sensors can be any type of sensors, such as, for example, but not limited to, position sensors, magnetic sensors, resistors, field-effect transistors, and/or any other type of sensors, and/or any combinations thereof. The current and/or voltage data may be gathered by the sensors and provided to the electronics 116. The electronics 116 may use the gathered data to determine location of the armature component 206 within the interior portion 208, and, based on the location, determine an intended operation of the impactor (e.g., forward, reverse, etc.). Once intended operation is ascertained, the electronics 116 may cause application of current to the coil(s) 204 to affect movement of the armature component 206 and hence, distal connector assembly 110. In some example implementations, the sensors may be configured to detect, interrogate, and/or cause the electronics 116 to determine armature component 206 position data continuously, periodically, during a specific movement of the armature component 206, dynamically (e.g., upon initiation of impact-related operations by the electronics 116), upon request, and/or using any other desired schedule.

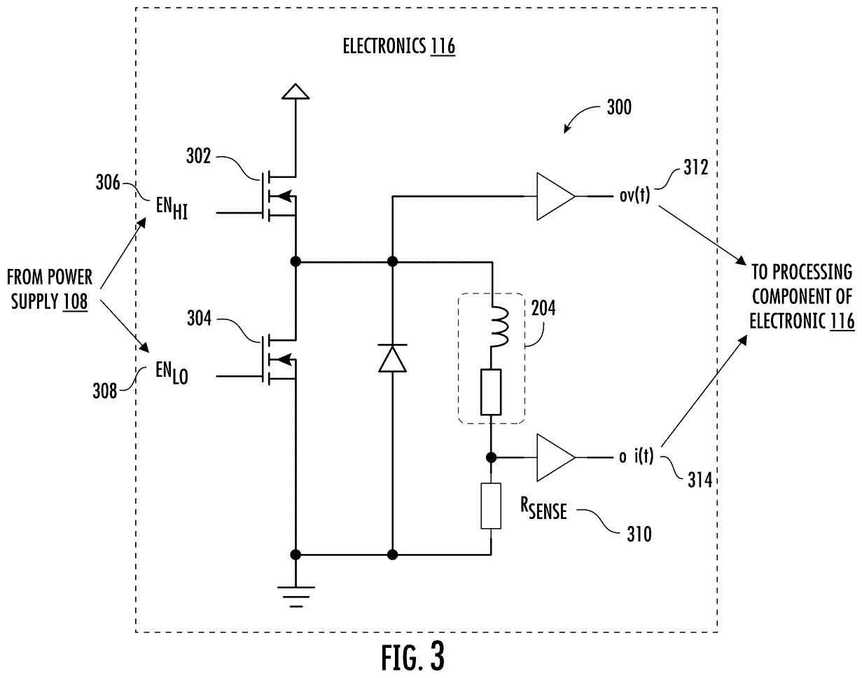

FIG. 3 illustrates an example controller 300 that may be incorporated into the electronics 116 of the impactor 100, in accordance with one or more features of the present disclosure. The controller 300 may be incorporated into the electronics 116 of the impactor 100. It may include one or more inputs (e.g., an input 302 on a high end of the coil and an input 304 on a low end of the coil), a sensing component 310, and one or more outputs (e.g., coil voltage output 312 and coil current output 314). The inputs 302, 304 may be coupled across the coil(s) 204. The sensing component 310 may be coupled in series with coil(s) 204.

The inputs 302, 304 may be any type of input components, such as, for example, one or more transistors and/or transistor circuits (e.g., metal-oxide field effect transistors (MOSFET), etc.) and/or any other type of input components that may be configured to receive one or more input signals, e.g., current, voltage, etc., from the power source 108. For example, the input 302 may be configured to receive a high coil energizing signal 306 for energizing one or more coil(s) 204. Likewise, the input 304 may be configured to receive a low coil energizing signal 308 for energizing one or more coil(s) 204. Energization of coil(s) 204 may be configured to generate an electromagnetic field surrounding the coil(s) 204 (as shown in FIGS. 2A and 2B). The strength of such electromagnetic field may be related to the strengths of the high coil energizing signal 306 and/or low coil energizing signal 308. The energizing signals 306, 308 may be current signals and/or voltage signals that may be received from power source 108. In some implementations, each of the coil(s) 204 may be energized individually and/or collectively. As can be understood, any desired way of energizing coil(s) 204 may be used.

Once the energizing signals are supplied across one or more coil(s) 204, the sensing component 310 may be configured to trigger generation of one or more of coil voltage output 312 (i.e., v(t)) and/or coil current output 314 (i.e., i(t)) signals. The coil(s) 204 voltage output signals 312 may be determined using any desired electronic components. These may, for example, include one or more voltage amplifiers and/or analog to digital converters (ADC), where the former may amplify an analog voltage signal generated in response to one or more signals 306, 308 and the latter may convert the amplified analog voltage into a digital signal for processing by the processing component of the electronics 116. Similarly, the coil(s) 204 current output signals 314 may be determined using one or more current amplifiers (which may amplify an analog current signal generated in response to one or more signals 306, 308) and/or ADCs (which may convert the amplified analog current into a digital signal to be processed by the processing component of the electronics 116).

The digitized coil voltage output 312 and the coil current output 314 may then be supplied to a processing component of electronics 116. The processing component may then determine position of the armature component 206 using the digitized signals 312, 314. In some implementations, multiple input signals 306, 308 may be supplied to the coil(s) 204 to determine multiple output signals 312, 314. One or more of the signals 306 and/or 308 may be the same and/or different from those previously supplied to the coil(s) 204. Use of different input signals 306, 308 may allow the electronics 116 to determine how coil(s) 204 are responding to changes in the input signals 306, 308.

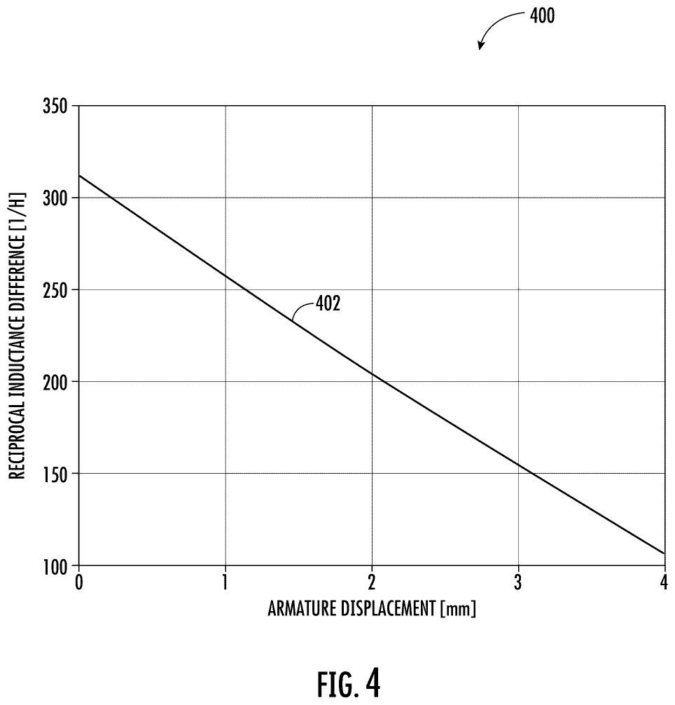

For each supplied input signal 306, 308, one or more of the output signals 312, 314 may be determined. Using the determined output signals 312, 314, the processing component of the electronics 116 may determine, using an energization time period for each coil(s) 204 (i.e., how long does it take to energize each coil 204), one or more of a mean voltage value and a best-fit current gradient. The mean voltage value and the current gradient may be used to determine one or more approximate coil inductance values for one or more or all coil(s) 204. The processing component of the electronics 116 may then use the approximate coil inductance values to determine a reciprocal inductance (i.e., an inverse of the inductance) for one or more or all coil(s) 204. A difference between reciprocal inductances between two or more coil(s) 204 may be determined by the processing component. Such difference may correspond to an approximate linear relationship to the position of the armature component 206. To do so, in some example, non-limiting implementations, the processing component may be configured to perform measurement of impactor system voltages and/or currents during an excitation of one or more coil(s) 204 (e.g., application of current to coil(s) 204). Then, for each coil, the processing component may determine an average voltage over one or more energization interval (e.g., sum of voltage samples divided by a number of voltage samples), determine an approximate first derivative of the current over the energization interval (e.g., determine a gradient using least-squares linear regression algorithm), determine a reciprocal inductance (e.g., current derivative/average voltage), determine a weighted sum of coil reciprocal inductances, and compare the weighted sum to a look-up table to determine position of the armature component 206. An example of a linear relationship 402 between the reciprocal inductance and the displacement 226 of the armature component 206 is shown in the plot 400 shown in FIG. 4. The processing component of the electronics 116 may then use the linear relationship 402 to determine an absolute position of the armature component 206 and/or compare it to a predetermined threshold to determine specific position of the armature component 206.

As discussed herein, if the processing component of the electronics 116 determines that the armature component 206 has been displaced (e.g., by the displacement 226 (e.g., 3 mm)) away from proximal wall 222 of the stationary electromagnetic housing 210, the processing component may indicate that a reverse movement by the impactor 100 is desired. Subsequently, the processing component may cause application of appropriate current (e.g., “reverse” current) to the coil(s) 204 to initiate reverse movement. Alternatively, if the processing component of the electronics 116 determines that the armature component 206 has been pressed against the proximal wall 222 of the stationary electromagnetic housing 210 (e.g., the displacement is 0 and/or below a predetermined threshold (e.g., 3 mm)), the processing component may indicate that a forward movement by the impactor is desired. Similarly, the processing component may then cause application of “forward” current to the coil(s) 204 to initiate forward movement. In some example, non-limiting implementations, the processing component may also generate an indication for display to the user of the impactor 100 stating that reverse or a forward movement is being initiated and/or performed. This may provide the user with an opportunity to cancel operation of the impactor, if necessary.

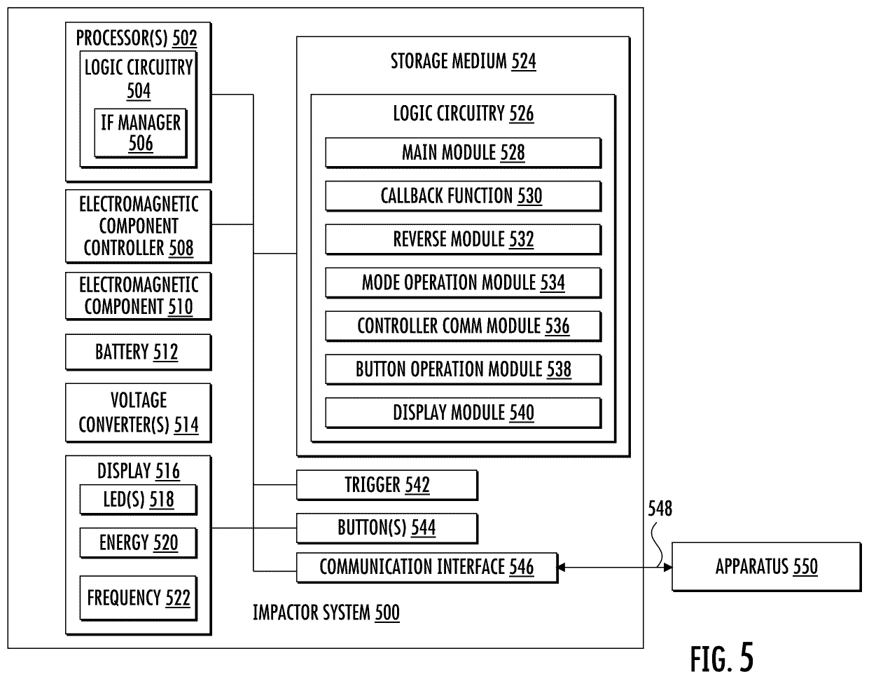

FIG. 5 is a block diagram of an exemplary orthopedic surgical instrument and/or impactor system 500, in accordance with one or more features of the present disclosure. The impactor system 500 may be similar to the impactor shown and described above in connection with FIGS. 1-3.

The impactor system 500 may combine with any suitable example of the systems, devices, and methods disclosed herein. The impactor system 500 may include one or more processor(s) 502, a non-transitory storage medium 524, an electromagnetic component controller 508, an electromagnetic component 510, a battery 512, a voltage converter(s) 514, a display 516, a trigger 542, button(s) 544, and a communication interface 546. The processor(s) 502 may include one or more processors, such as a programmable processor, a micro-controller unit (MCU), and/or the like. The processor(s) 502 may include processing circuitry to implement impactor logic circuitry 504 and logic circuitry 526.

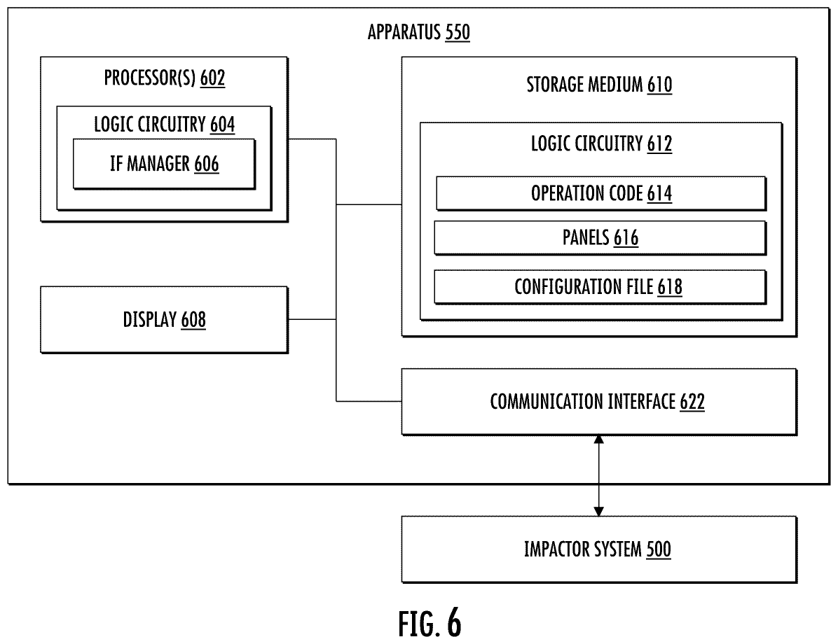

The processor(s) 502 may operatively couple with a non-transitory storage medium 524. The non-transitory storage medium 524 may store logic, code, and/or program instructions executable by the processor(s) 502 for performing one or more operations including the operations of the impactor logic circuitry 526. The non-transitory storage medium 524 may include one or more memory units (e.g., fixed or removable media or external storage such as a flash memory, secure digital (SD) card, random-access memory (RAM), read only memory (ROM), a flash drive, a hard drive, a solid-state drive (SSD) and/or the like). The memory units of the non-transitory storage medium 524 can store logic, code and/or program instructions executable by the processor(s) 502 to perform any suitable implementations of the current subject matter, as described herein. For example, the processor(s) 502 may execute instructions such as instructions of impactor logic circuitry 504 causing the electromagnetic component controller 508 to operate the impactor at an impact energy and/or frequency as determined using the above-described processes and/or selected by a user via trigger 542 and/or via apparatus 550 (as shown in FIG. 6).

The processor(s) 502 may include code for the impactor system 500 in memory within the processor(s) 502 and/or closely connected such as flash memory. The impactor logic circuitry 504 may represent code in or near the processor(s) 502 for execution by the processor(s) 502 and may include a user interface (IF) manager 506. The IF manager 506 may include code executing on the processor(s) 502 to detect and respond to user input as well as to detect the electromagnetic component controller 508 (such as, for example, a Maxon EPOS4 Controller) and establish communication with the electromagnetic component controller 508.

The user IF manager 506 may communicate with the electromagnetic component controller 508 to receive status information about the electromagnetic component 510 and to control operation of the electromagnetic component 510. For instance, all button presses of button(s) 544 and edit events may be posted to the IF manager 506 and processed in real-time. The IF manager 506 may communicate commands with the electromagnetic component controller 508 to execute in response to the user's actions via button presses, system states, and error conditions. The IF manager 506 may communicate alerts, warnings, and notifications to a user via the display 516 and or the apparatus 550 (as shown in FIG. 6) via the communication interface 546. Further, the IF manager 506 may also handle user's response to alerts.

The battery 512 may include any desired power source.

The voltage converter(s) 514 may include a DC-DC voltage converters to adjust the voltage of signals to various voltages required to operate the components of the impactor system 500 such as the processor(s) 502, the storage medium 524, and electromagnetic component controller 508, the display 516, the trigger 542, the button(s) 544, the communication interface 546, and/or the like.

The storage medium 524 may include a code for execution by the processor(s) 502 to operate the impactor system 500. If desired, the processor(s) 502 may copy code from the storage medium 524 to memory closer to the processor(s) 502 to facilitate faster execution of the code. For instance, the IF manager 506 may include code copied from the impactor logic circuitry 526 to memory closer to the processor(s) 502 for execution.

The impactor logic circuitry 526 may include code for operation of the impactor system 500 stored in hardware of the storage medium such as volatile or non-volatile memory in the storage medium 524. The impactor logic circuitry 526 may include a main module 528, a callback function 530, a reverse module 532, a mode operation module 534, a controller comm module 536, a button operation module 538, and a display module 540.

The main module 528 may include setup and loop functions. The setup function may run once at start-up and the loop function may run continuously afterwards. The setup function may attach interrupts that run when button(s) 544 are pressed on the user interface, initializes Timer1 which runs the trigger interrupt service routine (ISR), and initializes an impact delay for the electromagnetic component 510. The loop function allows the electromagnetic component 510 to operate in the user-desired mode when the trigger 542 is enabled and pulled. The loop function also handles showing the user that the trigger state is enabled via LED(s) 518 of the display 516 and/or via the apparatus 550 (shown in FIG. 6).

The callback function 530 may be, e.g., an ISR that runs every millisecond. In some example implementations, the callback function 530 may run periodically with at a time period of more than one millisecond or less than one millisecond.

The reverse module 532 may include functions to prepare to reverse the direction of translation of the electromagnetic component 510, direction change of the electromagnetic component 510, calculate impact delay of the impactor, and setup flutter time delays to set the frequency of impact while in flutter mode. These functions may switch the direction of translation of the electromagnetic component 510, reversing the electromagnetic component 510 to allow for bi-directional operation of the impactor, and may also determine the delay between reversals for controlling a frequency of impacts of the impactor in a flutter mode.

The mode operation module 534 may include the functions of position check, flutter check, and oscillation check functions which are called for normal/full-swing mode, high-frequency/flutter mode, and oscillation mode, respectively. Normal operation checks the position of the electromagnetic component 510 then calls the prepare to reverse function.

The controller communication module 536 may include the functions to enable electromagnetic component controller 508 functions, set current, set current direction, and disable the electromagnetic component controller 508 functions. These functions communicate to the electromagnetic component controller 508 whether or not to operate the electromagnetic component 510.

The button operation module 538 may include functions to handle setting user-desired frequency to operate the electromagnetic component 510 in addition to setting the operation mode and enabling the trigger 542. The functions may include energy plus to increase the energy of impact by the impactor, energy minus to increase the energy of impact by the impactor, frequency plus to increase the frequency of impacts by the impactor, frequency minus to decrease the frequency of impacts by the impactor, select operating mode to switch between available modes of operation (e.g., full-swing mode, flutter mode, or oscillation mode), and set trigger state to enable or disable the trigger 542. In some implementations, these functions may be accessed via the apparatus 550 (shown in FIG. 6) and/or the button(s) 544. In alternate implementations, a touch screen may be included in the display in lieu of or in addition to the button(s) 544.

The display module 540 may include functions handle the logic for displaying the amperage and frequency on the user interface. The functions may include energy display and frequency display.

The display 516 may include LED(s) 518 and numerical, alphanumeric, or graphical displays such as LED displays or liquid crystal displays (LCDs) to present a number representative of the energy 520 and frequency 522 selected for operation of the electromagnetic component 510. The button(s) 544 may include one or more buttons located in the display 516 and, in some implementations, adjacent to the energy 520 and frequency 522 displays to provide a user with an interface to increase and/or decrease the energy and/or frequency of the impact of the impactor on the forward and/or the reverse motion.

The trigger 542 may include a trigger or other button or switch that, when actuated, can cause the impactor system 500 to operate if the trigger 542 is enabled. If the trigger 542 is disabled, depressing the trigger 542 may not cause the impactor system 500 to operate.

The processor(s) 502 may couple to the communication interface 548 to communicate with an optional apparatus 550 via the communication interface 546. The communication interface 548 may include a wired or a wireless interface to communicatively coupled the impactor system 500 with the communication interface 548 shown in FIG. 6.

The communication interface 548 may optionally communicate user commands to and/or from the apparatus 550 to the impactor system 500 to operate the impactor system 500 via the functionality described in conjunction with the impactor system 500. In some implementations, the impactor system 500 may operate the electromagnetic component 510 (such as, for example, one or more components disposed within the housing 112, as shown in FIGS. 1 and 2A-B) in addition to configuring parameters of operation of the electromagnetic component 510 such as the operating current, the upper frequency bound, the lower frequency bound, the operating frequency, the mode of operation of the electromagnetic component 510, and/or the like. In some implementations, the communication interface 548 may communicate information about the operation of the impactor system 500 to the apparatus 550 such as the energy of operation, the frequency of operation, the mode of operation, events or alerts associated with the impactor system 500, and log information such as time and date of use, impact detections, encoder counts, and/or the like.

The communication interface 548 (and similarly, communication interface 622 shown in FIG. 6) may include circuitry to transmit and receive communications through a wired and/or wireless media such as an Ethernet interface, a wireless fidelity (Wi-Fi) interface, a cellular data interface, and/or the like. In some implementations, the communication interface 548 (and/or communication interface 622) may implement logic such as code in a baseband processor to interact with a physical layer device to transmit and receive wireless communications from apparatus 550. For example, the communication interface 548 (and/or communication interface 622) may implement one or more of local area networks (LAN), wide area networks (WAN), infrared, radio, Wi-Fi, point-to-point (P2P) networks, telecommunication networks, cloud communication, and the like.

FIG. 6 illustrates an exemplary computing apparatus 550, in accordance with one or more features of the present disclosure. The apparatus 550 may be a computing device that may be communicatively coupled with an orthopedic surgical instrument or impactor such as, impactor system 500 (e.g., as shown in FIG. 5). The apparatus 550 may be a computer in the form of a smart phone, a tablet, a notebook, a desktop computer, a workstation, or a server. The apparatus 550 can combine with any suitable example of the systems, devices, and methods disclosed herein. The apparatus 550 can include processor(s) 602, a non-transitory storage medium 610, communication interface 622, and a display 608. The processor(s) 602 may include one or more processors, such as a programmable processor (e.g., a central processing unit (CPU)). The processor(s) 602 may include processing circuitry to implement impactor logic circuitry 604 such as the impactor logic circuitry 504 shown in FIG. 5.

The processor(s) 602 may include memory such as flash memory to contain program code for execution by the processor(s) 602. In some implementations, the processor(s) 602 may have random access memory to contain a copy of code from flash memory or read only memory to facilitate faster execution of code. In some implementations, the processor(s) 602 may include cache to contain data for faster calculations or execution. In some implementations, the processor(s) 602 may include an impactor logic circuitry 604, which may include a user interface (IF) manager 606. The IF manager 606 may function as a state machine controlled by keypad inputs, internal events or alarms, boundary conditions, exceptions, and supervisory input to the IF manager 606. The IF manager 606 may process button presses and may update a main screen on the display 608 reflecting the state of the application.

Upon startup of the IF manager 606, a handler may be installed to detect the electromagnetic component controller 508 of the impactor system 500 and to establish communication with the electromagnetic component controller 508. In some implementations, the button presses of button(s) 544 and edit events may be posted to a panel in the display 608 and may be processed in real-time. Controller commands may be executed upon the user's actions via button presses, system states, and error conditions. Further, the IF manager 606 may implement alerts, warnings, and notifications and display the alerts, warnings, and notifications via the display 608. The IF manager 606 may also include code to handle the user's response to alerts, warnings, and notifications.

The processor(s) 602 may operatively couple with a non-transitory storage medium 610. The non-transitory storage medium 610 may store logic, code, and/or program instructions executable by the processor(s) 602 for performing one or more instructions including the impactor logic circuitry 612. The non-transitory storage medium 610 may include one or more memory units (e.g., fixed and/or removable media or external storage such as electrically erasable programmable read only memory (EEPROM), a secure digital (SD) card, random-access memory (RAM), a flash drive, solid-state drive, a hard drive, and/or the like). The memory units of the non-transitory storage medium 610 may store logic, code and/or program instructions executable by the processor(s) 602 to perform any suitable implementation of the methods described herein. For example, the processor(s) 602 may execute instructions such as instructions of impactor logic circuitry 612 causing one or more processors of the processor(s) 602 to communicate user commands to the impactor system 500 (as shown in FIG. 5) and/or to communicate events, alerts, operation parameters for the impactor system 500, and configurations.

The impactor logic circuitry 612 may include operation code 614, panels 616, and a configuration file 618. The operation code 614 may include functionality to set energy boundaries for operation of the impactor system 500, set frequency boundaries for operation of the impactor system 500, set an operating energy, set an operating frequency, set an impactor detection profile, set an operating mode (full swing, flutter, or oscillation), and/or the like.

The panels 616 may define graphical user interfaces for display of information and for receiving input parameters or configurations from a user. The configuration file 618 may include user selected parameters such as a controller with which to communicate, boundaries for energy (current), boundaries for frequency of impact, numbers of interrupts expected for push current and for pull current, and/or number of interrupts to receive to establish a frequency of impact.

The processor(s) 602 may couple to the communication interface 622 to transmit the data, code, or commands to and/or receive data, code, or commands from one or more external devices (e.g., a terminal, display device, a smart phone, a tablet, a server, or other remote device). The communication interface 622 includes circuitry to transmit and receive communications through a wired and/or wireless media such as an Ethernet interface, a wireless fidelity (Wi-Fi) interface, a Bluetooth interface such as a Bluetooth Low Energy (BLE) interface, a cellular data interface, and/or the like. In some implementations, the communication interface 622 may implement logic such as code in a baseband processor to interact with a physical layer device to transmit and receive wireless communications from the impactor system 500. For example, the communication interface 622 may implement one or more of local area networks (LAN), wide area networks (WAN), infrared, radio, Bluetooth, Wi-Fi, point-to-point (P2P) networks, telecommunication networks, cloud communication, and the like.

The processor(s) 602 may couple to the display 608 to display panels 616 for a user interface and/or other user interface items such as a message or notification via, graphics, video, text, and/or the like. In some implementations, the display 608 may include a display on a terminal, a display device, a smart phone, a tablet, a server, or a remote device.

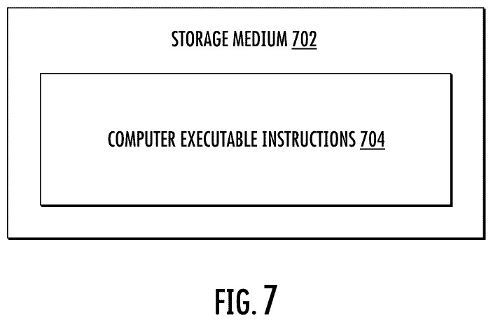

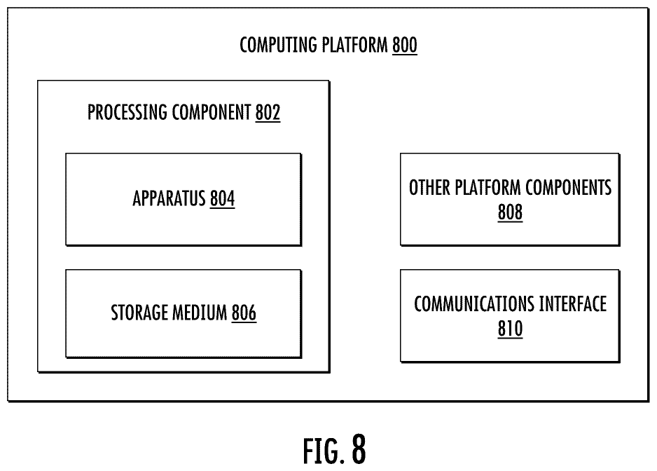

FIGS. 7-8 illustrate example implementations of a storage medium and computing platform for an orthopedic surgical instrument or impactor in accordance with one or more features of the present disclosure. FIG. 7 illustrates an example of a storage medium 702 to store impactor logic. Storage medium 702 may include an article of manufacture. In some examples, storage medium 702 may include any non-transitory computer readable medium or machine-readable medium, such as an optical, magnetic or semiconductor storage. Storage medium 702 may store various types of computer executable instructions 704, such as instructions to implement logic flows and/or techniques described herein. Examples of a computer readable or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions may include any suitable type of code, such as source code, compiled code, interpreted code, executable code, static code, dynamic code, object-oriented code, visual code, and the like. The examples are not limited in this context.

FIG. 8 illustrates an example computing platform 800. In some examples, as shown in FIG. 8, the computing platform 800 may include a processing component 802, other platform components 808 and/or a communications interface 810. According to some examples, computing platform 800 may be implemented in a computing device such as a server in a system such as a data center or server farm that supports a manager or controller for managing configurable computing resources as mentioned above. Further, the communications interface 810 may include a wake-up radio (WUR) and may be capable of waking up a main radio of the computing platform 800.

According to some examples, the processing component 802 may execute processing operations or logic for apparatus 804 described herein such as the impactor logic circuitry 504, 526, 604, 612, as illustrated in FIGS. 5-6. The processing component 802 may include various hardware elements, software elements, or a combination of both. Examples of hardware elements may include devices, logic devices, components, processors, microprocessors, circuits, processor circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. Examples of software elements, which may reside in the storage medium 806, may include software components, programs, applications, computer programs, application programs, device drivers, system programs, software development programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an example is implemented using hardware elements and/or software elements may vary in accordance with any number of factors, such as desired computational rate, power levels, heat tolerances, processing cycle budget, input data rates, output data rates, memory resources, data bus speeds and other design or performance constraints, as desired for a given example.

In some examples, other platform components 808 may include common computing elements, such as one or more processors, multi-core processors, co-processors, memory units, chipsets, controllers, peripherals, interfaces, oscillators, timing devices, video cards, audio cards, multimedia input/output (I/O) components (e.g., digital displays), power supplies, and so forth. Examples of memory units may include without limitation various types of computer readable and machine readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory), solid state drives (SSD) and any other type of storage media suitable for storing information.

In some examples, communications interface 810 may include logic and/or features to support a communication interface. For these examples, communications interface 810 may include one or more communication interfaces that operate according to various communication protocols or standards to communicate over direct or network communication links. Direct communications may occur via use of communication protocols or standards described in one or more industry standards (including progenies and variants) such as those associated with the PCI Express specification. Network communications may occur via use of communication protocols or standards such as those described in one or more Ethernet standards promulgated by the Institute of Electrical and Electronics Engineers (IEEE). For example, one such Ethernet standard may include IEEE 802.3-2012, Carrier sense Multiple access with Collision Detection (CSMA/CD) Access Method and Physical Layer Specifications, Published in December 2012 (hereinafter “IEEE 802.3”). Network communication may also occur according to one or more OpenFlow specifications such as the OpenFlow Hardware Abstraction API Specification. Network communications may also occur according to Infiniband Architecture Specification, Volume 1, Release 1.3, published in March 2015 (“the Infiniband Architecture specification”).

The computing platform 800 may be part of a computing device that may be, for example, a server, a server array or server farm, a web server, a network server, an Internet server, a workstation, a mini-computer, a main frame computer, a supercomputer, a network appliance, a web appliance, a distributed computing system, multiprocessor systems, processor-based systems, or combination thereof. Accordingly, functions and/or specific configurations of the computing platform 800 described herein, may be included or omitted in various implementations of the computing platform 800, as suitably desired.

The components and features of the computing platform 800 may be implemented using any combination of discrete circuitry, ASICs, logic gates and/or single chip architectures. Further, the features of the computing platform 800 may be implemented using microcontrollers, programmable logic arrays and/or microprocessors or any combination of the foregoing where suitably appropriate. It is noted that hardware, firmware and/or software elements may be collectively or individually referred to herein as “logic.”

It should be appreciated that the example computing platform 800 shown in the block diagram of FIG. 8 may represent one functionally descriptive example of many potential implementations. Accordingly, division, omission or inclusion of block functions depicted in the accompanying figures does not infer that the hardware components, circuits, software and/or elements for implementing these functions would necessarily be divided, omitted, or included in implementations.

One or more features of at least one example may be implemented by representative instructions stored on at least one machine-readable medium which represents various logic within the processor, which when read by a machine, computing device or system causes the machine, computing device, or system to fabricate logic to perform the techniques described herein. Such representations, known as “IP cores,” may be stored on a tangible, machine readable medium and supplied to various customers or manufacturing facilities to load into the fabrication machines that actually make the logic or processor.

The foregoing description has broad application. While the present disclosure refers to certain implementations, numerous modifications, alterations, and changes to the described implementations are possible without departing from the sphere and scope of the present disclosure, as defined in the appended claim(s). Accordingly, it is intended that the present disclosure not be limited to the described implementations. Rather these implementations should be considered as illustrative and not restrictive in character. All changes and modifications that come within the spirit of the current subject matter are to be considered within the scope of the disclosure. The present disclosure should be given the full scope defined by the language of the following claims, and equivalents thereof. The discussion of any implementation is meant only to be explanatory and is not intended to suggest that the scope of the disclosure, including the claims, is limited to these implementations. In other words, while illustrative implementations of the disclosure have been described in detail herein, it is to be understood that the inventive concepts may be otherwise variously embodied and employed, and that the appended claims are intended to be construed to include such variations, except as limited by the prior art. Unless otherwise defined, all technical terms used herein have the same meaning as commonly understood by one of ordinary skill in the art to which the disclosure belongs.

Directional terms such as top, bottom, superior, inferior, medial, lateral, anterior, posterior, proximal, distal, upper, lower, upward, downward, left, right, longitudinal, front, back, above, below, vertical, horizontal, radial, axial, clockwise, and counterclockwise) and the like may have been used herein. Such directional references are only used for identification purposes to aid the reader's understanding of the present disclosure. For example, the term “distal” may refer to the end farthest away from the medical professional/operator when introducing a device into a patient, while the term “proximal” may refer to the end closest to the medical professional when introducing a device into a patient. Such directional references do not necessarily create limitations, particularly as to the position, orientation, or use of this disclosure. As such, directional references should not be limited to specific coordinate orientations, distances, or sizes, but are used to describe relative positions referencing particular implementations. Such terms are not generally limiting to the scope of the claims made herein. Any implementation or feature of any section, portion, or any other component shown or particularly described in relation to various implementations of similar sections, portions, or components herein may be interchangeably applied to any other similar implementation or feature shown or described herein.

It should be understood that, as described herein, an “implementation” (such as illustrated in the accompanying Figures) may refer to an illustrative representation of an environment or article or component in which a disclosed concept or feature may be provided or embodied, or to the representation of a manner in which just the concept or feature may be provided or embodied. However, such illustrated implementations are to be understood as examples (unless otherwise stated), and other manners of embodying the described concepts or features, such as may be understood by one of ordinary skill in the art upon learning the concepts or features from the present disclosure, are within the scope of the disclosure. Furthermore, references to “one implementation” of the present disclosure are not intended to be interpreted as excluding the existence of additional implementations that also incorporate the recited features.

In one example, an apparatus may include an electromagnetic component including a stationary electromagnetic housing and a moving armature component, wherein the stationary electromagnetic housing includes a coil configured to receive an electric current resulting in generation of an electromagnetic field for triggering translation of the armature component within the stationary electromagnetic housing, wherein the electromagnetic field is configured force the armature component to translate in at least one direction; and a sensing component including at least one processor, the at least one processor is configured to determine an inductance, associated with the electromagnetic field, based on at least one of: at least one current output signal and at least one voltage output signal generated by the coil, and determine, based on the inductance, a position of the movable armature component within the stationary electromagnetic housing.

The apparatus may include wherein the inductance is determined based on a plurality of current output signals and/or a plurality of voltage output signals generated by the coil.

The apparatus may include wherein the position of the movable armature component is determined based on a reciprocal inductance.

The apparatus may include wherein the position of the movable armature component is determined based on a distance that the movable armature component translated within the stationary electromagnetic housing.

The apparatus may include wherein the at least one processor is configured to determine an intended direction of operation of the apparatus.

The apparatus may include wherein the intended direction includes at least one of: a first direction of operation of the apparatus and a second operation of the apparatus, wherein the second direction of operation is opposite of the first direction of operation.

The apparatus may include wherein upon the distance being greater than or equal to a predetermined threshold distance, the at least one processor is configured to determine that the intended direction is the first direction.

The apparatus may include wherein upon the distance being less than a predetermined threshold distance, the at least one processor is configured to determine that the intended direction is the second direction.

The apparatus may include a striker body configured to be coupled to the armature component and an object, wherein the at least one direction of translation movement of the armature component and the striker body is directly and/or indirectly dependent on a direction of the electric current applied to the coil.

The apparatus may include wherein a change in a direction of the electric current applied to the coil is configured to change the at least one direction of translation movement of the armature component and the striker component.

The apparatus may include wherein the coil includes a first coil configured to receive the electric current for translating the armature component in a first direction, and a second coil configured to receive the electric current for translating the armature component in a second direction.

The apparatus may include wherein the at least one direction includes a forward impact direction, a reverse impact direction, a combination of forward and reverse impact directions, and any combination thereof.

The apparatus may include wherein the object includes at least one of: a tool, an implant, and any combination thereof.

The apparatus may include wherein the translation movement of the striker body is configured for positioning an implant in a bone and/or removal of the implant from the bone.

The apparatus may include wherein the coil is configured to receive one or more current pulses to trigger translation of the armature component and the striker body.

The apparatus may include wherein the one or more current pulses include a high current pulse and a low current pulse, wherein the low current pulse is smaller than the high current pulse.

In one example, a method for operating a surgical impactor tool, the surgical impactor tool including an electromagnetic component having a stationary electromagnetic housing and a moving armature component, wherein the stationary electromagnetic housing includes a coil configured to receive an electric current resulting in generation of an electromagnetic field for triggering translation of the armature component within the stationary electromagnetic housing, wherein the electromagnetic field is configured to force the armature component to translate in at least one direction, the method may include determining, using at least one processor of the surgical impactor tool, an inductance, associated with the electromagnetic field, based on at least one of: at least one current output signal and at least one voltage output signal generated by the coil; determining, using the at least one processor, based on the inductance, a position of the movable armature component within the stationary electromagnetic housing; and operating, using the at least one processor, the surgical impactor tool based on the determined position.

The method may include wherein the determining the inductance includes determining the inductance based on a plurality of current output signals and/or a plurality of voltage output signals generated by the coil.

The method may include wherein the determining the position of the movable armature component includes determining at least one of: a reciprocal inductance, a distance that the movable armature component translated within the stationary electromagnetic housing, or any combination thereof.

The method may include determining an intended direction of operation of the surgical impactor tool, wherein the intended direction includes at least one of: a first direction of operation of the surgical impactor tool and a second operation of the surgical impactor tool, wherein the second direction of operation is opposite of the first direction of operation.

The method may include at least one of determining, upon the distance being greater than or equal to a predetermined threshold distance, that the intended direction is the first direction; or determining, upon the distance being less than a predetermined threshold distance, that the intended direction is the second direction.

In addition, it will be appreciated that while the Figures may show one or more implementations of concepts or features together in a single implementation of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one implementation can be used separately, or with another implementation to yield a still further implementation. Thus, it is intended that the present subject matter covers such modifications and variations as come within the scope of the appended claims and their equivalents.

As used herein, an element or step recited in the singular and proceeded with the word “a” or “an” should be understood as not excluding plural elements or steps, unless such exclusion is explicitly recited. It will be further understood that the terms “includes” and/or “comprising,” or “includes” and/or “including” when used herein, specify the presence of stated features, regions, steps, elements and/or components, but do not preclude the presence or addition of one or more other features, regions, integers, steps, operations, elements, components and/or groups thereof.

The phrases “at least one,” “one or more,” and “and/or,” as used herein, are open-ended expressions that are both conjunctive and disjunctive in operation. The terms “a” (or “an”), “one or more” and “at least one” can be used interchangeably herein.

Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

The foregoing discussion has been presented for purposes of illustration and description and is not intended to limit the disclosure to the form or forms disclosed herein. For example, various features of the disclosure are grouped together in one or more implementations or configurations for the purpose of streamlining the disclosure. However, it should be understood that various features of the certain implementations or configurations of the disclosure may be combined in alternate implementations or configurations. Moreover, the following claims are hereby incorporated into this detailed description by this reference, with each claim standing on its own as a separate implementation of the present disclosure.

What is claimed:

1. An orthopedic surgical apparatus, comprising:
an electromagnetic component including a stationary electromagnetic housing and a moving armature component, wherein the stationary electromagnetic housing includes a coil configured to receive an electric current resulting in generation of an electromagnetic field for triggering translation of the armature component within the stationary electromagnetic housing, wherein the electromagnetic field is configured to force the armature component to translate in at least one direction; and
a sensing component including at least one processor, the at least one processor is configured to
determine an inductance, associated with the electromagnetic field, based on at least one of: at least one current output signal and at least one voltage output signal generated by the coil, and
determine, based on the inductance, a position of the movable armature component within the stationary electromagnetic housing.

2. The apparatus of claim 1, wherein the inductance is determined based on a plurality of current output signals and/or a plurality of voltage output signals generated by the coil.

3. The apparatus of claim 1, wherein the position of the movable armature component is determined based on a reciprocal inductance.

4. The apparatus of claim 1, wherein the position of the movable armature component is determined based on a distance that the movable armature component translated within the stationary electromagnetic housing.

5. The apparatus of claim 1, wherein the at least one processor is configured to determine an intended direction of operation of the apparatus.

6. The apparatus of claim 5, wherein the intended direction includes at least one of: a first direction of operation of the apparatus and a second operation of the apparatus, wherein the second direction of operation is opposite of the first direction of operation.

7. The apparatus of claim 6, wherein upon the distance being greater than or equal to a predetermined threshold distance, the at least one processor is configured to determine that the intended direction is the first direction.

8. The apparatus of claim 6, wherein upon the distance being less than a predetermined threshold distance, the at least one processor is configured to determine that the intended direction is the second direction.

9. The apparatus of claim 1, further comprising a striker body configured to be coupled to the armature component and an object, wherein the at least one direction of translation movement of the armature component and the striker body is directly and/or indirectly dependent on a direction of the electric current applied to the coil.

10. The apparatus of claim 9, wherein a change in a direction of the electric current applied to the coil is configured to change the at least one direction of translation movement of the armature component and the striker component.

11. The apparatus of claim 9, wherein the coil includes a first coil configured to receive the electric current for translating the armature component in a first direction, and a second coil configured to receive the electric current for translating the armature component in a second direction.

12. The apparatus of claim 9, wherein the at least one direction includes a forward impact direction, a reverse impact direction, a combination of forward and reverse impact directions, and any combination thereof.

13. The apparatus of claim 9, wherein the object includes at least one of: a tool, an implant, and any combination thereof.

14. The apparatus of claim 9, wherein the translation movement of the striker body is configured for positioning an implant in a bone and/or removal of the implant from the bone.

15. The apparatus of claim 9, wherein the coil is configured to receive one or more current pulses to trigger translation of the armature component and the striker body.

16. A method for operating a surgical impactor tool, the surgical impactor tool including
an electromagnetic component having a stationary electromagnetic housing and a moving armature component, wherein the stationary electromagnetic housing includes a coil configured to receive an electric current resulting in generation of an electromagnetic field for triggering translation of the armature component within the stationary electromagnetic housing, wherein the electromagnetic field is configured to force the armature component to translate in at least one direction, the method comprising:
determining, using at least one processor of the surgical impactor tool, an inductance, associated with the electromagnetic field, based on at least one of: at least one current output signal and at least one voltage output signal generated by the coil;
determining, using the at least one processor, based on the inductance, a position of the movable armature component within the stationary electromagnetic housing; and
operating, using the at least one processor, the surgical impactor tool based on the determined position.

17. The method of claim 16, wherein the determining the inductance includes determining the inductance based on a plurality of current output signals and/or a plurality of voltage output signals generated by the coil.

18. The method of claim 16, wherein the determining the position of the movable armature component includes determining at least one of: a reciprocal inductance, a distance that the movable armature component translated within the stationary electromagnetic housing, or any combination thereof.

19. The method of claim 16, further comprising determining an intended direction of operation of the surgical impactor tool, wherein the intended direction includes at least one of: a first direction of operation of the surgical impactor tool and a second operation of the surgical impactor tool, wherein the second direction of operation is opposite of the first direction of operation.

20. The method of claim 19, further comprising at least one of
determining, upon the distance being greater than or equal to a predetermined threshold distance, that the intended direction is the first direction; or
determining, upon the distance being less than a predetermined threshold distance, that the intended direction is the second direction.
